(19) United States
(12) Patent Application Publication
(10) Pub. No.: US 2026/0315000 A1
(43) Pub. Date: Oct. 15, 2026

(54) METHOD FOR MONITORING AND TREATING A DISORDER

(71) Applicant: Northwestern University, Evanston, IL (US)

(72) Inventor: Rod Passman, Evanston, IL (US)

(73) Assignee: Northwestern University, Evanston, IL (US)

(21) Appl. No.: 19/116,609

(22) PCT Filed: Sep. 28, 2023
(86) PCT No.: PCT/US2023/033945

§ 371 Date: Mar. 28, 2025

Related U.S. Application Data
(60) Provisional application No. 63/412,104, filed on Sep. 30, 2022.

(51) Int. Cl. A61B 5/00; A61B 5/332; A61B 5/361

(52) U.S. Cl. CPC A61B 5/4839; A61B 5/332; A61B 5/361; A61B 5/681; A61B 5/747

(57) ABSTRACT

The present disclosure is generally directed to a method of monitoring and treating a disorder and a system for monitoring and treating a disorder. The method includes monitoring a subject, determining a time period that the risk of an adverse event, such as a risk of stroke provoked by a cardiac arrhythmia (such as atrial fibrillation AFib), is elevated, and administering a medical treatment or notifying a patient to begin a treatment, such as starting to take daily a direct acting oral anticoagulant DOAC. The system includes a device to monitor the patient that is communicatively coupled to a server. The server receives monitoring data from the device and transmits the data to a storage database. In response to the device detecting data indicative of a meeting or exceeding a predetermined threshold, the server sends a notification to the patient via the device.

### CROSS-REFERENCE TO RELATED PATENT APPLICATIONS

This application claims priority to U.S. provisional application Ser. No. 63/412,104 filed on Sept. 30, 2022, the entire contents of which are incorporated by reference herein.

### STATEMENT OF GOVERNMENT SUPPORT

This invention was made with government support under grant UG3HL165065 awarded by the National Institutes of Health (NIH). The government has certain rights in the invention.

### TECHNICAL FIELD

The present disclosure related general to a method for monitoring and treating a disorder. More specifically, the present invention relates to the identification of a marker that exceeds a threshold in real time and the medical intervention provided.

### BACKGROUND

The following discussion is provided to aid the reader in understanding the disclosure and is not admitted to describe or constitute prior art thereto.

Medical intervention often takes place after the onset of signs or symptoms regarding a disorder occurs or after an adverse medical event. Administering a treatment, such as a therapeutic intervention or an invasive procedure, after an adverse event occurs results in a greater stress and burden placed on the patient and may allow for irreversible morbidity. The patient may be faced with a longer and more strenuous recovery time due to the lack of monitoring a disorder in real time and intervening prior to the onset of irreversible disease. Furthermore, the patient may face a longer hospital stay due to the invasive nature of the procedure, or the need of monitoring a therapeutic intervention in a clinical setting, as a result of the lack of timeliness for administering healthcare. This ultimately places a larger financial burden on the patient and the health care system. As the average lifespan increases there is a need for preventative care and more personalized patient care to reduce the burden on healthcare systems worldwide, especially in growing aging populations with increased comorbidities that further complicate many diagnosis and disorders.

Care administered after an adverse event occurs is considered reactionary. In contrast to preventive care, reactionary care exposes the patient to the morbidity and mortality risk of the index event. Furthermore, reactionary care increases the financial burden on the patient and healthcare system. Even in the absence of debilitating injury or fatality, reactionary care leaves the patient with the fear of another adverse event occurring in the future and knowing the course of action they may once again have to endure. Preventative care for many diseases is highly cost-effective but can sometimes expose the patient to the risks and costs associated with preventive therapy. In some cases, adverse events associated with preventative care may prove more serious than the disease-associated event it is being used to prevent.

Atrial fibrillation (AF) is an abnormal rhythm in which the upper chambers of the heart beat rapidly and irregularly. Atrial fibrillation is the most common heart rhythm disorder that affects millions of people worldwide and may often have little to no symptoms. Regardless of symptoms, atrial fibrillation increases the risk of forming blood clots. The propensity towards the formation of blood clots increases the patient's risk of experiencing an atrial fibrillation related stroke by 500%. Atrial fibrillation related strokes have shown to be severely debilitating and more fatal than non-atrial fibrillation related strokes. Therefore, an atrial fibrillation diagnosis weighs heavily on patients because of the increased risk of a debilitating or fatal stroke due to an arrhythmia that may be silently occurring in their everyday life.

Historically, stroke prevention in atrial fibrillation has been treated preventatively. For example, a patient would traditionally receive an electrocardiogram (ECG) to confirm an arrhythmia (an irregular heart rhythm). If the patient had other additional stroke risk factors (i.e. advanced age, hypertension, diabetes, etc.), the physician would then prescribe the patient an anticoagulant (e.g., an oral anticoagulant taken daily) to be taken regardless of whether the atrial fibrillation was constantly present (permanent AF) or intermittently present (e.g., paroxysmal, or persistent AF). Overall, atrial fibrillation increases the risk of stroke by as much as 500%. Studies have shown that daily oral anticoagulation can reduce the risk of atrial fibrillation stroke by about two-thirds. However, the use of daily oral anticoagulants also introduces the risk of major and minor bleeding which can be life-threatening. The risk of bleeding is often considered less critical compared to the risk of a severely debilitating or fatal atrial fibrillation related stroke, so most patients are willing to comply with taking a daily oral anticoagulant. The burden and risk of taking a daily oral anticoagulant may reduce the patient's quality of life due to concerns about bleeding events and is mentally taxing and financially burdensome. Continuous anticoagulation places a significant financial burden on the patient as well as the healthcare system while introducing further health risk that may be life threatening.

Therefore, physicians and patients are constantly working to balance the benefits and risks of preventative care in order to mitigate the risk of an atrial fibrillation related stroke that would require reactionary care. Furthermore, the effect of the treatment plan on the patient's quality of life and the financial burden imposed adds additional challenges to treating an atrial fibrillation diagnosis.

The use of wearable, implantable, and external personal monitoring systems to track and diagnose medical events or markers has been discussed in previous literature; however, current devices fall deficient for some interventions for various reasons. The IMPACT clinical trial investigated atrial arrhythmia monitoring to guide anticoagulation in patients who had received implanted contemporary dual-chamber cardioverter defibrillators (ICDS) and biventricular pacemakers, known as cardiac resynchronization devices (CRTs).¹ Patients enrolled in this trial had implantable devices for other medical indications that were capable of detecting atrial fibrillation or flutter. Data regarding atrial fibrillation events transmitted via a wireless telemetry device occurred nightly. The patient was required to plug the wireless telemetry device in near there bedside to allow data to flow wirelessly from their implantable device, to the wireless telemetry device, and finally to a physician monitored database. However, as shown in this trial, implantable devices have the capability to monitor disorders such as atrial fibrillation, but the devices are highly invasive, which introduces further risk, and requires indications of other medical disorder for the utilization of this method. ICDs and CRTs communicative with wireless telemetry devices also add another restraint to a patient's day to day activity, thus reducing their quality of life. Furthermore, this method is lacking timeliness of treatment and are not patient-facing. The transmission of data only at night means that events that occurred in the morning would not be reported to the physician and communicated to the patient for potentially 24 hours or longer after the event occurred. Sending data at night further means that the physician may not review the data until the next workday, adding more time between the occurrence of the medical event and the time in which intervention has begun.

Other studies have also used other implantable devices, like insertable cardiac monitors (ICMs) and permanent pacemakers (PPMs), to guide anticoagulation treatment. Unlike the IMPACT clinical trial that used implantable devices requiring direct leads to the heart, the REACT pilot study utilized leadless ICMs to guide anticoagulation. Furthermore, unlike the IMPACT study, the devices used in the REACT study were intended to be used for the primary disorder which was atrial fibrillation.^{1,2} Patients were instructed to manually transmit data to study centers by waving a secondary external device near the ICM that was placed in their chest near their heart. Similar to the IMPACT study, the REACT study involved invasive and costly devices and lacked timeliness of data transmission.

Similar to the IMPACT study and the REACT pilot study, the TACTIC study further utilized ICDs and PPMs to guide anticoagulation through remote transmission of data biweekly.^{1,2,3} The TACTIC study further emphasizes the invasiveness and deficiencies of data transmission and medical intervention of the devices and methods shown in the IMPACT and the REACT study. Therefore, although these devices and implemented methods make strides towards personalized methods, they are still insufficient, leaving a growing patient population with a critical unmet medical need.

Although these studies have shown positive results, the use of implantable devices for continuous monitoring of atrial fibrillation is not scalable. For example, implantable devices are currently on the market for tens of thousands of dollars. Furthermore, the cost of staffing healthcare workers to continuously monitor the data being retrieved from the implantable device is also extremely costly.

While great strides have been made in the world of implantable devices, such as increased battery life, they are still invasive. The invasive nature of implantable devices presents more risk factors and potential complications to the patient such that they may decline this opportunity if continuous monitoring. Implantable monitoring devices also place a large cost burden on the healthcare system due to the high cost of device implantation and remote monitoring. Furthermore, implantable devices lack direct patient-facing communication with the device. While nearly all implantable devices currently on the market can automatically send patient data to a physician, the data is not reported in real time nor is it patient facing. In regard to many medical interventions, timeliness is critical in preventing a serious adverse event. Therefore, implants are lacking the real-time data transmission to the patient or health care provider and thus no opportunity is afforded that allows for the timely analysis needed for preventative and personalized patient care.

Furthermore, external devices may include devices that the patient selectively activates to monitor a condition. An example of an external device may include an application for a mobile device that detects heart rhythms or an ECG through sensors. External devices are an improvement to implantable devices because they are noninvasive and do not interfere with the patient's daily life. However, external devices lack the ability to passively monitor a medical condition since the patient must actively initiate the recording of the physiologic event.

Furthermore, wearable devices for monitoring a disorder may include devices such as watches, patches, and straps. Wearable devices, such as watches or patches, have become increasingly popular. The current market price of these devices is a small fraction of an implant making them much more accessible to people around the world. However, early wearables lacked battery life for prolonged continuous monitoring and did not have any algorithms for sensing abnormal heart rhythm including atrial fibrillation. These deficiencies in wearable devices results in less than optimal monitoring conditions that may leave critical gaps in patient data needed for prescribing proper medical intervention.

The deficiencies herein described above have left a critical gap in patient care which ultimately reduces a patient's quality of life. Previous implantable devices on the market, as previously described, are not only invasive, but add a new element of patient fear regarding financial burden, placement complications, and lack timeliness of communicating detected markers. Furthermore, historically wearable devices, as previously described, lacked the battery-life to continuously monitor patients, which is a critical element in identifying the frequency and duration of medical events in order to provide the highest quality of care over the lifetime of the individual. Thus continuous monitoring outside of serious medical disorders outside of a healthcare setting to guide immediate care and intervention with high levels of accuracy was simply not possible.

The risk of major complications from several disorders, such as atrial fibrillation, is mitigated if the current standard of care is followed. For atrial fibrillation, that includes anticoagulant therapy to reduce the risk of stroke which is the most feared consequence of the disorder. However, concerns of side effects including major and minor bleeding events that are associated with the prescribed treatment plan reduce compliance, leaving the individual susceptible to events that will impact morbidity and mortality. Also, many medical events or markers associated with a disorder, including atrial fibrillation events, occur silently without the patient feelings or knowing they have occurred. Therefore, on top of device deficiencies, patient fear regarding side effects and unknown medical events further reduces treatment compliance and quality of life. These factors contribute to a significant under usage of these medications where only 55% of eligible patients are prescribed anticoagulants, and almost half of those patients stop the medication within two years. Therefore the combination of continuous remote monitoring and device guided medical intervention provides an attractive potential treatment option but have not been coupled into treatments plans thus far.

Newer wearable devices now have a battery life of 60 hours and longer, meaning patients are able to wear their device for a full day such that they are monitored during active hours and times of rest. The longer battery life of noninvasive wearable devices allows for a more accurate assessment of specific markers related to health disorders. Furthermore, wearable devices such as smart watches are patient-facing and provide real time feedback on biological processes that can be shared with a healthcare provider if necessary.

Previous studies have shown feasibility and high compliance rates even with invasive monitoring devices that require patient engagement that still interrupt daily life. Patients have shown willingness to comply in return for reassurance that they are working with their physician for personalized care as to mitigate their risk of future health complications or adverse events.^{1,2,3,4}

Furthermore, regardless of the limitations of heart rhythm monitoring devices, the onset of action of the only previously available drug for stroke prevention (e.g., Coumadin, etc.) made acute recognition of the onset of atrial fibrillation moot as an effective stroke prevention strategy would take several days to take effect and expose the individual to a heightened risk of stroke during this time.

However, the development of rapid onset pharmaceuticals in some areas of medicine has provided an opportunity for an effective treatment to be rendered in close proximity to the onset of a biologic event. For example, direct acting anticoagulants, referred to as DOACs, are formulated to provide therapeutic anticoagulation within hours of ingestion compared to traditional anticoagulants, such as warfarin, that typically take days to reach therapeutic onset. Beyond the therapeutic benefits, newly formulated DOACs carry a lower major bleeding risk than traditional anticoagulants, albeit at an increased cost. The current standard of care for stroke prevention in AF is for at-risk patients to be prescribed chronic oral anticoagulants as a form of preventive care. For example, if a patient is diagnosed with atrial fibrillation, the patient would likely be prescribed an oral anticoagulant to take daily due to the increased risk of stroke associated with atrial fibrillation. Given that AF episodes are often asymptomatic, chronic anticoagulation is prescribed regardless of whether the patient is in AF continuously or has rare or even absent episodes either spontaneously or as the result of a medical intervention. This treatment approach exposes the individual to the risks and costs of chronic anticoagulation even during long periods of a normal rhythm when the benefits may be altogether absent.

DOACs are type II transmain inhibitors which are all rapid onset pharmaceuticals. DOACs currently on the market for use in reducing stroke risk in atrial fibrillation include, but are limited to dabigatran (known as Pradaxa), rivaroxaban (Xarelto), apixaban (known as Eliquis), edoxaban (known as Savaysa), and betrixaban (known as Bevyxxa). Furthermore, some DOACs may also include but are not limited to (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl] sulfamoyl] acetic acid (YM-466 or YM-60828), or eribaxaban (PD 0348292), carbamimidoy 1-2-hydroxy-phenyl) 445-(2,6-dimethyl-piperidin-1-yl)-pentyl]-3-oxo-3,4-dihydro-quinoxaline-6-carboxylic acid (PD0313052), or analogues compositions⁵.

Administering medical intervention, such as prescribing a pharmaceutical, may prevent one adverse event while increasing the risk of another adverse event. Therefore, the patient may be faced with the difficult decision of deciding to comply with one mode of recommended medical intervention at the risk of inducing a different, sometimes life-threatening, adverse event. For most patients the benefits outweigh the risks, but the potential for harm imposes both a mental and physical burden on the patient and impacts decision making.

Because of the development of rapid onset therapeutics, like DOACs, patients may no longer need to be prescribed an anticoagulant preventatively if an AF alert system is in place to notify the individual to take appropriate therapy only in response to a biologic process or biomarker, even when it creates no obvious symptoms. Physicians may now prescribe DOACs when medical events, such as atrial fibrillation, are present and the patient will receive therapeutic benefits within hours, lowering their risk of adverse events while minimizing exposure to the inherent risks of the medical therapy during periods of no benefit. The patient can be intermittently administered the DOAC for a limited time (i.e. 4 weeks), meaning the patient only takes the prescribed DOAC when they are at an increased risk for an adverse medical event, such as an atrial fibrillation associated stroke which may occur when atrial fibrillation burden is high. When the risk of an adverse event, such as stroke, is once again lowered (e.g. no atrial fibrillation event detected within a 4 week period), the patient can stop taking the prescribed DOAC, thus removing the risk of major or minor bleeding when there is no need for preventative care. The intermittent administration of DOACs could minimize the risk of a main adverse event, mitigate the risk of a secondary adverse event, lower overall healthcare costs, all while increasing the patient's quality of life.

However, administration of rapid onset therapeutics intermittently would require that the targeted medical event, or marker, be identified. In some disorders, the identification of the medical event or associated marker may require continuous or near-continuous monitoring over prolonged time periods. Therefore, the identification of the medical event or marker traditionally would only occur within a medical setting where a patient can be monitored for hours to days at a time or in the presence of an implanted device that allows for remote monitoring. Therefore, patients are not receiving the medical benefit of “fast-acting” therapeutics due to the lack of ability to identify and monitor medical events or marker remotely and in a timely manner that indicate a need for medical intervention.

The combination of the lack of timeliness of detecting a health marker on top of the lack of timeliness of administering a medical intervention, such as prescribing a therapeutic drug, has resulted in less than optimal patient care. Even with great strides made in pharmaceutical development, such that targeted pharmaceuticals can take notable action in a patient within hours, prematurely or unnecessarily prescribing them can still induce unwanted adverse health events as an associated complication.

Adverse health events associated with long-term pharmaceutical use may reduce patient compliance due to the fear of inducing an adverse event while trying to prevent another adverse event.

Therefore, there is a need for a continuous or near continuous monitoring system that allows for detection of health markers and determines risk based on the extent of the health marker. The determined risk allows for proper medical intervention to be taken in a manner that reduces risk of a main adverse health event while mitigating the risk of an associated adverse health event that may stem from the medical intervention itself. Furthermore, a patient-facing system that does not interrupt daily activity would improve a patient's quality of life through personalized treatment while keeping the patient informed in an easy to understand and timely manner. Treatment of medical conditions in a point-of-care setting provides personalized medicine to patients at a fraction of the cost.

Specifically, there is a need for a point-of-care device for use within a continuous or near continuous monitoring system for the detection atrial fibrillation. Furthermore, there is a need for the point of care device to reliably monitor a patient remotely, such that the patient's everyday life is not interfered with and there is no reduction to their quality of life. A point-of-care device with the aforementioned capabilities would allow for more personalized healthcare that provides a plurality of benefits to the patient including treatment plans that reduce the risk of atrial fibrillation stroke while mitigating the potential of major or minor bleeding associated with the current standard of care.

### SUMMARY

This disclosure relates generally to an embodiment for monitoring a health marker according to a predetermined threshold set for the health marker and calculating a time period over which the health marker identified presents an increased risk of an adverse event such that medical intervention will be administered. Furthermore, an example of this embodiment is a method for detecting atrial fibrillation comprising a wearable atrial fibrillation monitoring device (e.g. an atrial fibrillation sensing smart watch).

One embodiment is a method of treating a disorder comprising monitoring a subject in order to detect a marker that exceeds a predetermined threshold, determining a time period during which the subject is at a greater risk of an adverse event relating to the disorder based on the detected marker that exceeded the predetermined threshold, and administering a medical intervention to the subject for the calculated period of time during which the greater risk of the adverse event is present.

One embodiment is a system of monitoring and treating a disorder comprising a monitoring device. According to some embodiments, the monitoring device is configured to connect to a personal electronic device. In other embodiments the personal monitoring device is a standalone device, thus able to transmit data freely on its own. The personal monitoring device is configured to detect a level of a marker for a disorder in a subject in real time, determine a time period that the level of the marker is detected, determine if the time period meets or exceeds a pre-determined threshold time period during which the subject is at risk for an adverse event associated with the disorder, and communicate to the patient to begin a medical intervention (e.g., a predetermined prescribed medication).

Furthermore, in some embodiments, the monitoring device is configured to communicate the detected level of the marker and the determined time period to a server. The server may be configured to receive the marker and time period data from the device and further configured to, responsive to the marker exceeding the pre-determined threshold, notify the patient to begin a treatment, and receive an acknowledgement from the patient.

One embodiment is a method of monitoring and treating a disorder comprising monitoring a subject in order to detect whether a marker exceeds a predetermined threshold; determining a time period during which the subject is at a greater risk of an adverse event relating to the disorder based on the detected marker; and administering a medical intervention to the subject for the calculated period of time during which the greater risk of the adverse event is present.

### BRIEF DESCRIPTION OF THE DRAWINGS

FIG. 1 shows a high level overview flow chart of one exemplary embodiment of the monitoring and treatment system for a disorder.

FIG. 2 shows a high level overview flow chart of another exemplary embodiment of the monitoring and treatment system for a disorder.

FIG. 3 shows a high level overview flow chart of one exemplary embodiment of the monitoring and treatment system for a disorder. The flow chart shows communication between a patient and a physician in which the patient has the option to directly contact the physician regarding the notification sent regarding a health event that has occurred.

FIG. 4 shows a high level overview of one exemplary embodiment of how a wearable or implantable monitoring device may communicate with a physician and a patient through the patient's personal mobile device.

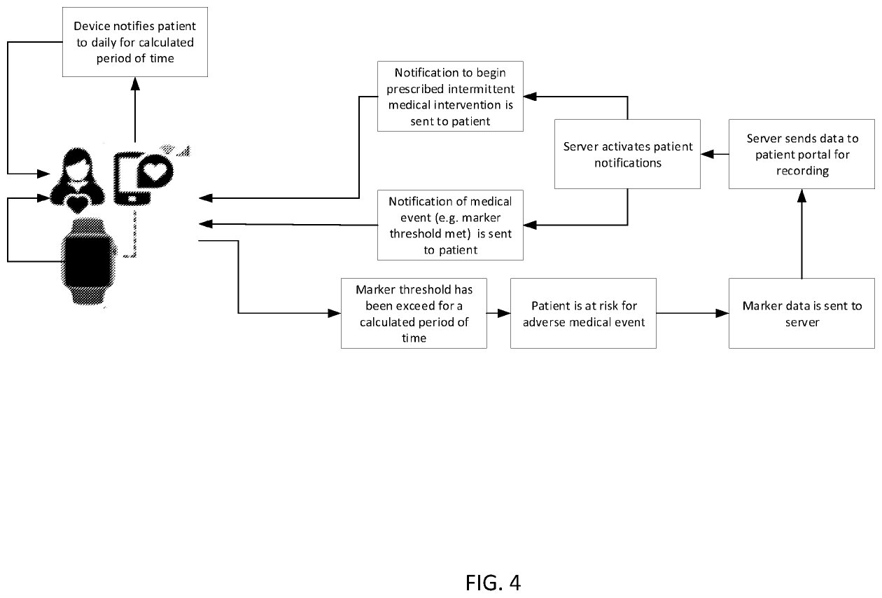

FIG. 5 shows a high level overview of another exemplary embodiment of how a wearable or implantable monitoring device may communicate with a physician and a patient through the patient's personal mobile device.

FIG. 6 shows an embodiment for which a monitoring system may be advantageous to patient care. This figure shows generally accepted association between calculated risk scores and an adverse event according as it applies to one example. This example is related to the association of patient health demographics, such as age, weight, and other health history with known atrial fibrillation used to calculate CHA2DS2-VASC scores and the association between this score and the risk percentage of a stroke associated with the score. This figure is one exemplary embodiment of risk stratification of a patient population to determine high versus low risk patient populations for a main adverse event (e.g. stroke).

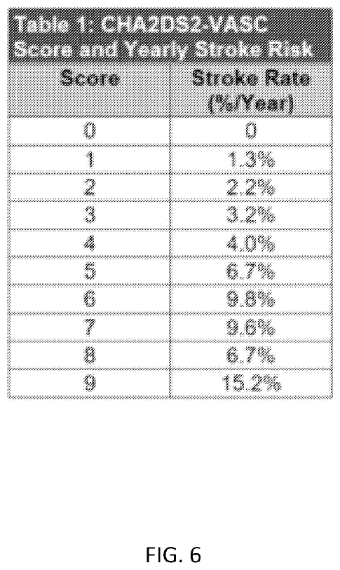

FIG. 7 depicts a flow chart for inclusion into a continuous monitoring program according to one exemplary embodiment. This figure depicts the inclusion or exclusion into a continuous monitoring program for a disorder by determining risk prior. This embodiment depicts protocol to be followed to ensure a patient is not at an extreme high risk in which the mitigation of a secondary risk may be far less superior to only targeting the mitigation of a main adverse event.

FIG. 8 shows a push notification sent to a subject regarding the detection of a marker according to one exemplary embodiment. This embodiment depicts a notification regarding a marker related to atrial fibrillation. However, the notification for a disorder is not limited to this depiction and may include a set of instructions based on a prescribed medical intervention plan determined by a physician.

FIG. 9 shows a system of communication between a patient and a physician for notifying and recording the detection of a marker associated with a health event according to one exemplary embodiment. This embodiment is a high level flow chart that may include, but is not limited to the steps and actions herein depicted. This embodiment may include further steps and actions taken that may steps and actions that are specific for each patient or tailored to a patient's specific need. The flow chart of communication and action according to this embodiment may be customizable such that the patient may receive a personalized care plan.

FIG. 10 depicts a monitoring and treatment system according to one exemplary embodiment. The system may include a communication server, and a health record data base. The communication center may be able to, but is not limited to, sending push notifications to a patient to remind them of a prescribed medical intervention plan once a marker of a health event has been detected and communicate data with a physician and the health record database.

FIG. 11 depicts a monitoring and treatment system according to another exemplary embodiment. The system may include a communication server, and a health record data base. The communication center may be able to, but is not limited to, sending push notifications to a patient to remind them of a prescribed medical intervention plan once a marker of a health event has been detected and communicate data with a physician and the health record database.

FIG. 12 shows an interaction between a patient and a device for continuous monitoring according to one exemplary embodiment. This embodiment includes, but is not limited to a noninvasive wearable device, such as a smartwatch. Further this embodiment depicts a monitoring and treatment system for atrial fibrillation but may be representative of a monitoring and treatment system for various disorders.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

The expression “comprising” means “including, but not limited to.”

As used herein, the term “subject” is used interchangeably with “patient” and is referring to a human.

A “medical intervention” is administering any action with the intention of preventing an adverse medical event.

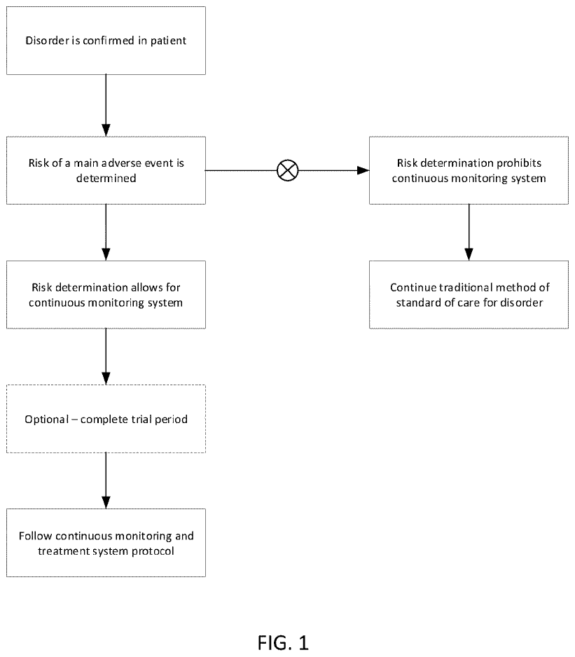

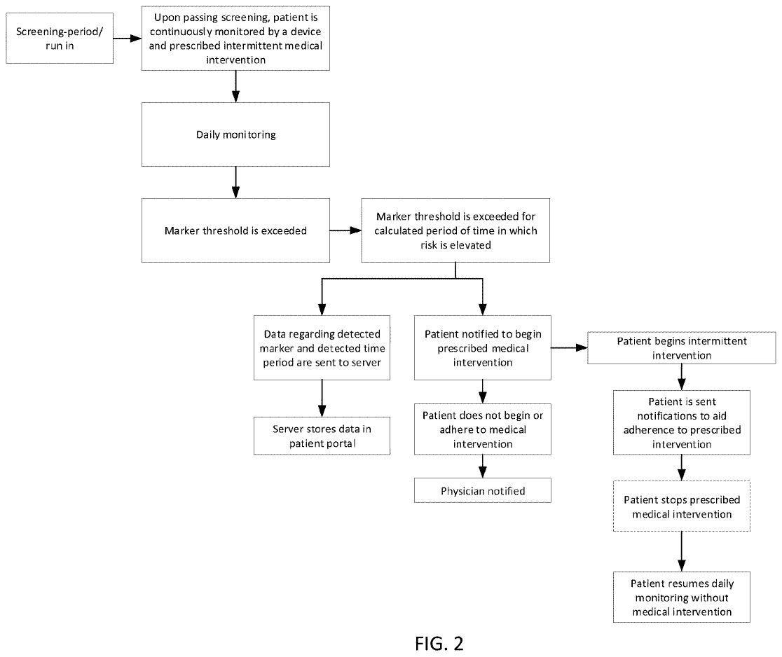

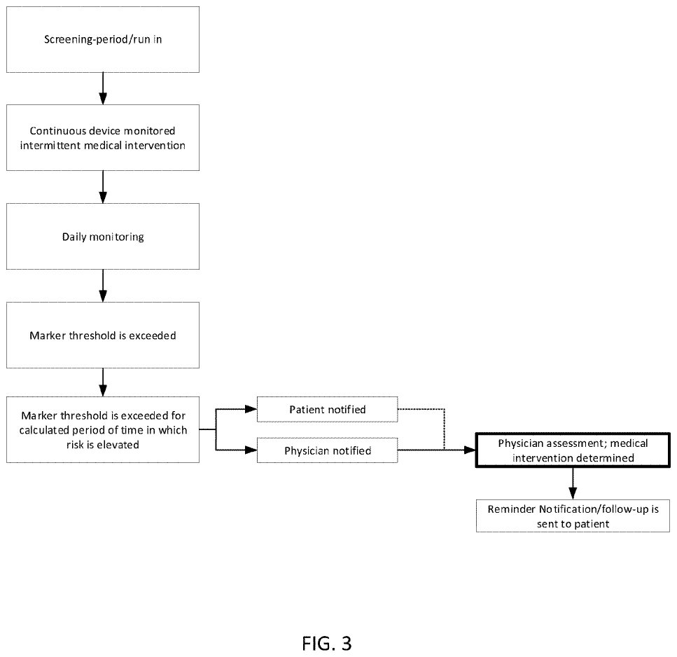

An “effective amount” is an amount sufficient to effect beneficial or desired results. An effective amount can be administered in one or more administrations, applications or dosages based on patient specific metrics. The following method may refer generally to the exemplary embodiments of FIGS. 1-3.

### Methods

The present disclosure is directed to a novel method of continuous monitoring of a health marker for a disorder such that medical intervention can be taken in a point of care setting.

For example, a patient may have a known history of a health disorder. This may include, but is not limited to cardiovascular disorders, diabetes care/continuous glucose monitoring, x, y, and z. The associated health disorder may require administering a drug, orally or intravenously, so that the patient is in a therapeutic range to mitigate the risk of a main adverse health event. For example, the administration of anticoagulant may reduce the risk of blood clot. Furthermore, the health disorder may require more invasive intervention, which may include but is not limited to a surgery or interventional operation to prevent an adverse health event.

Once the health disorder has been identified, an associated risk may also be identified. For example, the standard of care may include administering a therapeutic amount of a drug for a specified time period. However, this standard of care may be held for patients of all risk level. Therefore, for example a patient of low risk of a main adverse health event may be administered the same medical intervention as a patient of high risk of the main adverse health event associated with a given health disorder. The medical intervention may pose in itself a risk of a secondary adverse health event. For example, medical intervention (e.g. administering a therapeutic drug) to a patient of low risk of the main adverse health event may greatly increase their risk of the secondary adverse health event.

According to this exemplary embodiment, by continuously (e.g. greater than 14 hours a day) monitoring a health marker (e.g. pulse, heart rate, activity level, etc.) associated with a disorder, medical intervention (e.g. prescription drugs, surgical intervention, etc.) may be administered. For example, the monitoring device may include but is not limited to, a wearable smartwatch, a smart ring, an implantable pacemaker, an implantable defibrillator, or a wearable patch. For example, known (e.g. generally accepted) thresholds regarding a disorder correlated to a specific patient demographic may be used as a comparison data point for real time data collected on a monitoring device.

According to another exemplary embodiment, a physician, or other qualified clinician, can set a predetermined threshold on the monitoring device (e.g. wearable or implantable). For example, the physician may determine a threshold based on a patient's past health history and current health indication, such as weight, age, and other confounding variables present that may be associated with the identified disorder. If the monitoring device (e.g. a smart watch) detects that a marker exceeds a predetermined threshold, a notification can be sent out to the patient via a personal smart device (e.g. a smart watch, smart phone, etc.) Furthermore, the notification can be sent directly to the patient's physician.

According to some embodiments, the notification regarding the marker exceeding the predetermined threshold may be sent to other consented recipients. For example, the patient may consent for a family member (e.g. a spouse, child, etc.) to also be notified in the event that a marker has exceeded a predetermined threshold.

Furthermore, a time period may be calculated regarding the duration of the detection of a marker exceeding a predetermined threshold. For example, if the marker is detected at an elevated level (e.g. exceeding the predetermined threshold) by the continuous monitoring device, an associated risk level may be calculated. The associated risk level can be assessed by a physician in order to determine the administration of a medical intervention.

According to an exemplary embodiment of this system, the physician may be able to administer care at the point of a marker being detected exceeding a predetermined threshold for a calculated period of time such that the patient is at an elevated risk of an adverse medical event occurring. For example, the physician may be able to prescribe a therapeutic drug to the patient (e.g. the patient can fill the prescription) such that the patient can have the prescribed medication on hand in the event that the marker of interest exceeds a predetermined threshold and their physician communicates to them to begin taking the prescribed medication for a prescribed duration of time to mitigate the risk of a main adverse health event. For example, according to an exemplary embodiment of the system, a notification may be sent to the patient via a smart device (smart watch, smart phone, etc.) that the marker has exceeded a predetermined threshold for a calculated time period imposing a risk to the patient's health. A second notification may be sent via the smart device (e.g. smartwatch, smartphone, etc.) from their physicians notifying the patient to begin taking the prescribed mediation (e.g. oral drug) for a prescribed period of time (e.g. 30 days, 60 days, etc.)

In some embodiments, the medical intervention may include other means such as surgical intervention or more invasive treatment based on the assessment made by the patient's physician and the communication between the patient and their physician. For example, the patient may receive a first notification regarding a detection of a marker exceeding a predetermined threshold for a calculated period of time associated with a risk. The patient then may choose to directly contact their physician (e.g. phone call) to discuss this first notification and the prevailing treatment options. For example, the patient may consult with their physician and determine that the best medical intervention for their personal health is surgical intervention. In this embodiment, the system for monitoring and administering a medical intervention emphasizes the communication aspect between a patient and their physician in a real time point of care setting.

Furthermore, according to an exemplary embodiment of this system, in order to maximize compliance, the system may require the patient to acknowledge the second notification (e.g. notification regarding a specific medical intervention). For example, if a physician provides a second notification regarding a medical intervention that may include administering a therapeutic drug regimen, the patient may be required to acknowledge (e.g. physically tap a screen to accept the notification) receipt of the second notification to begin treatment (e.g. the medical intervention). According to this embodiment, if the patient does not acknowledge the second notification, further notification may be sent to the patient via their personal smart device (e.g. smartwatch, smartphone, etc.). For example this further notification may be a plurality of push notifications. Furthermore, this may include a plurality of notifications in the form of cellular phone calls in attempt to contact the patient regarding administration of the medical intervention.

In some exemplary embodiments, the system of monitoring and administering medical intervention regarding a disorder may include the monitoring device automatically sending data collected regarding the detected marker levels and the type of medical intervention administered to a data storage media (e.g. patient healthcare record database).

### EXAMPLES

The present example regarding a monitoring and treatment system for a disorder relates generally to a method of monitoring a health marker and administering medical intervention related to the health marker detected. Furthermore, this method can be applied to monitoring and administering treatment for disorders such as atrial fibrillation.

For example, atrial fibrillation is the most common sustained heart rhythm disorder that affects millions of Americans and places a large financial burden on the U.S. Healthcare system. The prevalence of atrial fibrillation is predicted to more than double by the year 2050 as the American population continues to age and atrial fibrillation risk factors, such as diabetes and obesity, continue to rise. Furthermore, atrial fibrillation is associated with congestive heart failure, dementia, premature death, and notably, a 3-to-5 fold higher risk of stroke, as of which are costly to the U.S. healthcare system. Because risk of stroke increases with age, about a quarter of all strokes in patients over the age of 80 are due to atrial fibrillation of which are more likely to be severely debilitating or fatal than those not attributed to atrial fibrillation.

To combat the risk of stroke, patients are put on continuous oral anticoagulants (OAC), however this put the patient at a sustained and significant risk for major and minor bleeds even when sinus rhythm appears to be normal and risk of stroke is low. Therefore, on average, only about half of patients that qualify for continuous oral anticoagulants are prescribed them and even then as much as 60% of patients prescribed continuous oral anticoagulants discontinue treatment.

Therefore, in this exemplary method of monitoring and treating a health disorder in a point-of-care setting, an objective is to provide a method for monitoring atrial fibrillation and administering time-delimited direct oral anticoagulant (DOAC) therapy treatment when risk of stroke is high due to the presence of atrial fibrillation.

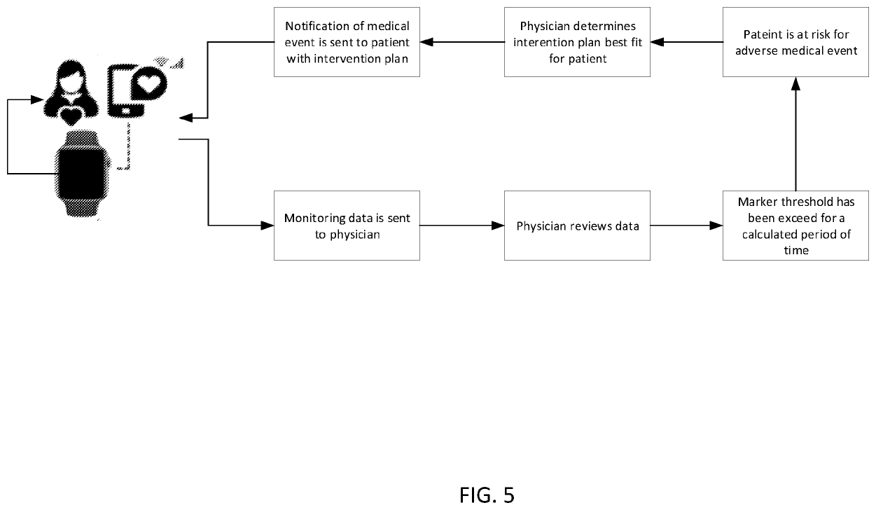

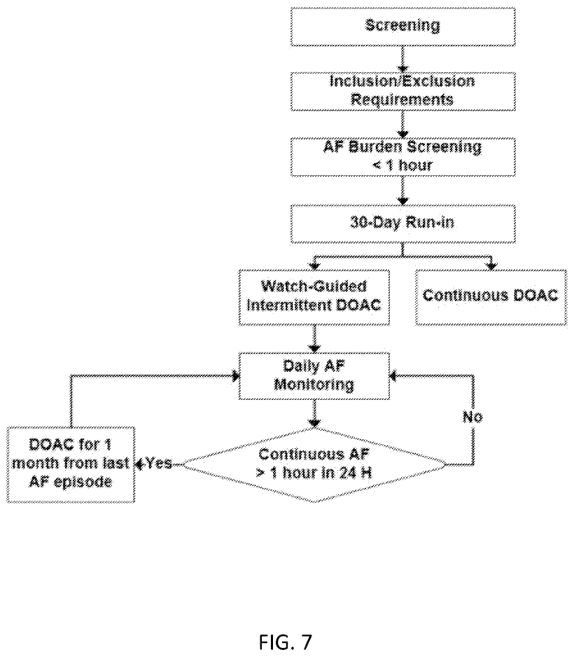

Now referring generally to FIG. 7. FIG. 7 is one exemplary embodiment of a monitoring and treatment system for a disorder including, but not limited to, atrial fibrillation. According to this example, a patient may be eligible for continuous monitoring of atrial fibrillation after completing a “run-in period.” For example, the run in period may include a screening period where the patient may be continuously monitored while on the patients current treatment plan (e.g. old standard of care plan, non-personalized plan, generalized plan etc.). For example referring to atrial fibrillation, the atrial fibrillation screening period may include a period of time (e.g. 30 days) of continuous monitoring (e.g. via a wearable device and potentially a second wearable device) while still continuing the patients current treatment plan (e.g. oral anticoagulant) to confirm that no marker (e.g. atrial fibrillation events) are detected during that time period. According to this embodiment, if the disorder (e.g. atrial fibrillation) is not detected, then the patient may be considered a candidate for the continuous monitoring and treatment system. For example, the patient may be monitored passively by a noninvasive wearable device, such as a smart watch. The smartwatch may passively collect health data regarding the disorder (e.g. atrial fibrillation) such as heart rate, pulse, and activity. The passive monitoring nature of a noninvasive wearable device may allow for real-time data monitoring of a health marker, or plurality of health markers, that may relate to an adverse health event while not interfering with the patient's life. For example, the patient may no longer have the burden of taking a medication (e.g. an oral anticoagulant) daily. Furthermore, for example, the embodiment of the monitoring and treatment system according to FIG. 5 may increase the quality of patient care by reducing the burden of daily medication and reducing a secondary adverse medical event that may be associated with the daily medication.

In the event that a health marker (e.g. atrial fibrillation) is detected for a prolonged period of time (e.g. greater than an hour) in which the calculated risk of an adverse medical event occurring (e.g. atrial fibrillation related stroke) is increased, the patient may be advised by a physician to medically intervene. For example, a monitoring and treatment system for atrial fibrillation, may include a notification server that may communicate with the patient via their monitoring device (e.g. smartwatch) and personal mobile device. For example, the physician may send the patient a notification to via the communication server to their monitoring device and personal mobile device to medically intervene in the form of intermittently restarting direct oral anticoagulation for defined period of time (e.g. 30 days) to ensure the risk of an adverse medical event (e.g. stroke) relating to atrial fibrillation is reduced.

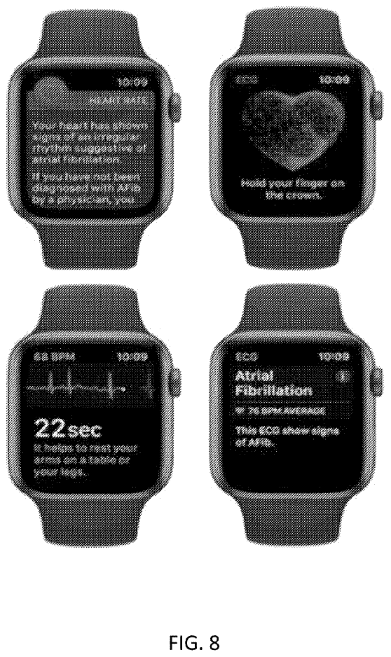

FIG. 8 is a depiction of a notification that can be sent to a patient regarding the detection of a health marker (e.g. atrial fibrillation) via a monitoring device according to one exemplary embodiment. This embodiment includes, but is not limited to, a notification sent to the patient on a noninvasive wearable smart watch. The notification may also be sent to the patient's personal mobile device. According to some embodiments, the notifications sent to the patient from the communication server may also be configured to send a notification to a family member (e.g. spouse, child, etc.) if the patient provides consent. For example, this may increase compliance in the event that the patient may be asked by their physician to medically intervene following the detection of a health marker for a prolonged period of time indicating an increased risk.

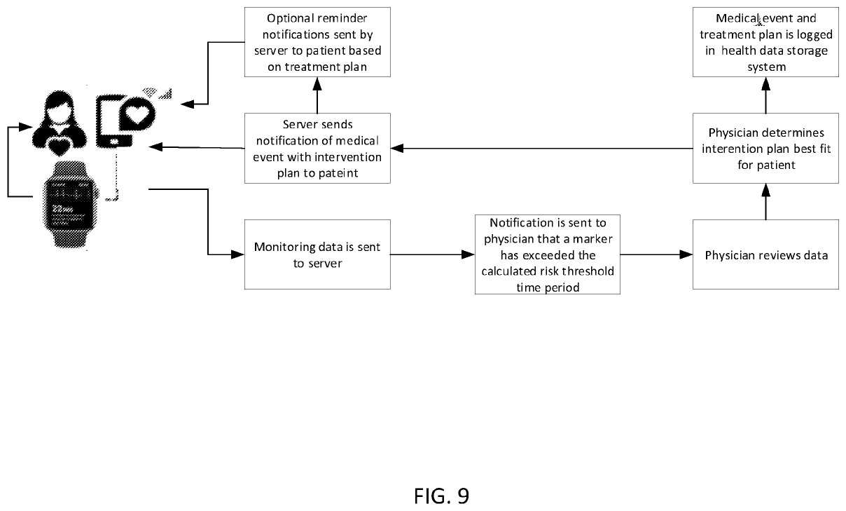

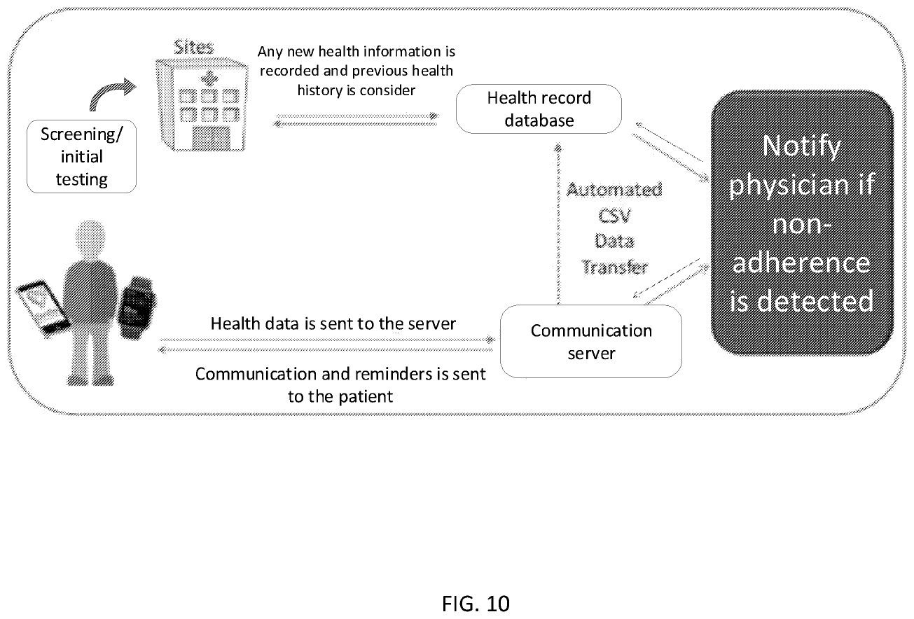

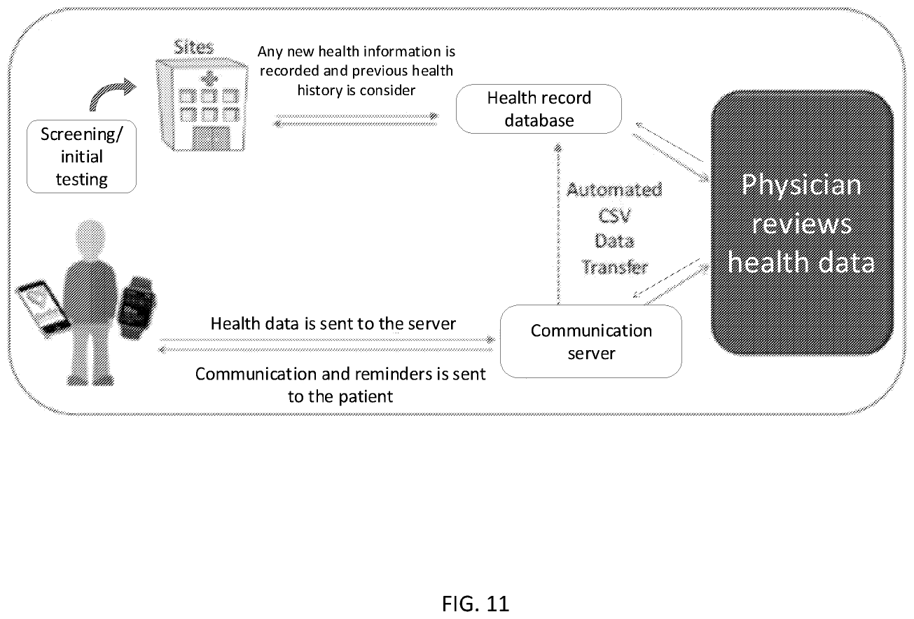

Furthermore, the communication of the monitoring and treatment system for a disorder may be depicted in the exemplary embodiments of FIG. 9 and the exemplary embodiment of FIG. 10 and FIG. 11. FIG. 10-11 are high level flow chart depiction of the interaction between a patient, a monitoring device worn by the patient, a communication server, and a physician. The embodiment of FIG. 9 further stems from the embodiment of FIG. 10-11 to depict the connection to a health storage database. The communication depicted in FIG. 9 may be incorporated into the monitoring and treatment system depicted in FIG. 10-11 in some embodiments. For example, the health record database may connect to the communication server such that data collected and transmitted by a patient's monitoring device may be automatically sent to and recorded in the patient's personal section the health record database. For example, according to this embodiment, the automatic communication between the communication server and the health record database may facilitate timely and accurate data procurement, a critical aspect of health care.

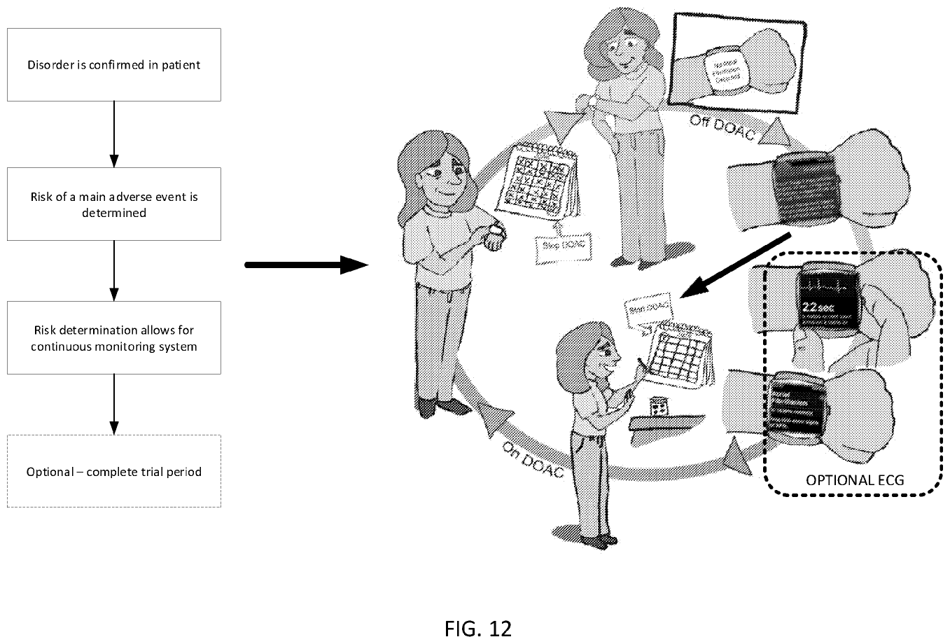

Now referring generally to the schematic drawing of FIG. 12 depicting one exemplary embodiment of a continuous monitoring and treatment system for a disorder. This embodiment depicts the monitoring and treatment system of a disorder that may require intermittent medical intervention. According to some embodiments, the medical disorder may first be diagnosed by a physician within a clinical setting. For example, the physician may confirm the medical disorder, such as atrial fibrillation, using clinical technology, such as an electrocardiogram (ECG). Furthermore, in some embodiments the physician may suggest a trail period or run in period where the patient takes a DOAC for a period of time. Instead, in other embodiments the trial period may not be necessary. For example, for a disorder, such as atrial fibrillation, an elevated risk of an adverse medical event associated with atrial fibrillation may be calculated over a predetermined period of time. Therefore, once the predetermined period of time has elapsed, the risk of an associated adverse medical event may be reduced and the need for medical intervention may no longer be present. Furthermore, once the calculated predetermined time period of elevated risk of an adverse event has elapsed, the risk of a secondary adverse medical event related to the medical intervention may be present. Therefore, according to this example, it may be advantageous for a monitoring and treatment system to be intermittent such that the risk of a main adverse event and a secondary adverse event are both lowered. For example, a disorder, such as atrial fibrillation, may be detected by a marker for a prolonged period of time such that the calculated risk of stroke related to the marker associated with an atrial fibrillation event is elevated. The physician may receive the data on a health marker from a monitoring device, such as a smart watch, and issue a notification via a communication server to the patient's monitoring device to medically intervene. In this example, the calculated time period of risk may be 30 days, such that the patient may need medical intervention (e.g. anticoagulation, ablation procedure, etc.) over that time period. For example, the patient may be administered a direct oral anticoagulant (e.g. a medical intervention) to take daily over the 30 day period to reduce the risk of an atrial fibrillation stroke. After the 30 day risk period has elapsed, the patient may be sent a second notification from the physician via the communication server to the monitoring device to stop taking the direct oral anticoagulant (e.g. the medical intervention) so that the risk of a secondary adverse medical event (e.g. major or minor bleeding) associated with the direct oral anticoagulant is lowered now that the risk of the main adverse health event (e.g. stroke) is reduced.

The novel method and system of monitoring and treating a disorder, such as atrial fibrillation, as described in the present application provides a significant benefit to the patient and the overall healthcare system. The present disclosure provides a method and system of personalized point-of-care to the patient, thus providing the first patient-facing method as opposed to the historically physician-facing methods.

While certain embodiments have been illustrated and described, it should be understood that changes and modifications can be made therein in accordance with ordinary skill in the art without departing from the invention in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed invention.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting. Any recited method can be carried out in the order of events recited or in any other order which is logically possible.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### CITED REFERENCES

1. David T. Martin, Randomized trial of atrial arrythmia monitoring to guide anticoagulaiton in patient with implanted defibrilaator and cardiac resynchronization devices, 36 European Heart Journal, 1660-1668 (2015).
2. Rod Passman, Targeted Anticoagulation for Atrial Fibrillation Guided by Continuous Rhythm Assessment With an Insertable Cardiac Monitor: The Rhythm Evaluation for Anticoagulation With Continuous Monitoring (REACT.COM) Pilot Study, Journal of Cardiovascular Electrophysiology, 264-270 (2015).
3. Rachel M. Kaplan, Stroke Risk as a Function of Atrial Fibrillation Duration and CHA₂DSₓ-VASc Score, 140 Circulation, 1639-1646 (2019).
4. Rod Passman, “Pill-in-Pocket” Anticoagulation for Atrial Fibrillation Fiction, Fact, Foolish?, 143 Circulation, 2211-2213 (2021)
5. Yan John, Anticoagulant compounds and methods and devices for their use, Patent Application No. US20220039976A1, February, 2022.

Other embodiments are set forth in the following claims.

What is claimed:

1. A method of treating a disorder comprising:
monitoring a subject in order to detect whether a marker exceeds a predetermined threshold,
determining a time period during which the subject is at a greater risk of an adverse event relating to the disorder based on the detected marker that exceeded the predetermined threshold, and
administering a medical intervention to the subject for the calculated period of time during which the greater risk of the adverse event is present.

2. The method of claim 1, wherein the disorder is monitored by a personal monitoring device that is at least one of a wearable device, an external device, and an implantable device configured to connect to a personal mobile device.

3. The method of claim 2, wherein the personal monitoring device intermittently and passively assesses the subject to detect the marker.

4. The method of claim 3, wherein the subject wears the monitoring device for an average time period of at least 14 to 24 hours a day, wherein the average time period includes active hours and/or resting hours.

5. The method of claim 3, wherein the subject wears the monitoring device for a near continuous amount of time.

6. The method of claim 1, wherein the medical intervention is intermittently administered, the method further comprising:
responsive to determining whether the marker exceeds the predetermined threshold, administering the medical intervention to the subject for the calculated period of time during which the greater risk of the adverse event is present;
responsive to completing the calculated period of time, ending the medical intervention; and
responsive to determining the marker does not exceed the predetermined threshold, continue to monitor the subject without administering treatment.

7. A system of monitoring and treating a disorder comprising:
a device configured to:
connect to a personal electronic device,
detect a level of a marker for a disorder in a subject in real time,
determine a time period that the level of the marker is detected,
determine if the time period meets or exceeds a pre-determined threshold time period during which the subject is at risk for an adverse event associated with the disorder, and
communicate the detected level of the marker and the determined time period to a server; and
a server configured to receive the marker and time period data from the device and further configured to:
responsive to the marker exceeding the pre-determined threshold, notify the patient to begin a treatment, and
receive an acknowledgement from the patient.

8. The system of claim 7, wherein the server is configured to notify the subject with a plurality of push notifications, including sending an SMS text message to the subject after a push notification is repeated once without acknowledgment from the subject.

9. The system of claim 8, wherein the server is further configured to contact a health center if the plurality of push notifications are not acknowledged by the subject.

10. The system of claim 9, wherein the device further comprises a heart rhythm detector configured to determine a time period during which the subject is at greater risk of having an adverse cardiovascular event based on the detection of an irregular heart rhythm that exceeds a pre-determined threshold.

11. The system of claim 10, wherein the health center is further configured to:
responsive to detecting non-adherence of wearing the device:
contact the patient; and
contact the physician.

12. The system of claim 7, wherein the server is further configured to:
notify a physician to review the marker and the time period data,
notify a subject to review the marker and the time period data and prepare to receive instructions from a physician, and
communicate a treatment plan from the physician to the subject.

13. A method of monitoring and treating a disorder comprising:
monitoring a subject in order to detect a marker that exceeds a predetermined threshold;
determining a time period during which the subject is at a greater risk of an adverse event relating to the disorder based on the detected marker; and
administering a medical intervention to the subject for the calculated period of time during which the greater risk of the adverse event is present.

14. The method of claim 13, wherein the server is further configured to:
notify a physician if the marker exceeds the pre-determined threshold and provide the time period calculation;
prompt the physician to review the marker and the time period detected to determine the medical intervention;
notify the subject; and
communicate a treatment plan from the physician to the subject.

15. The method of claim 13, wherein the disorder is atrial fibrillation.

16. The method of claim 15, wherein atrial fibrillation is monitored by an atrial fibrillation detecting device worn during a time of activity and a time of rest and configured to:
detect the marker of atrial fibrillation.

17. The method of claim 15, wherein atrial fibrillation is monitored by an implantable atrial fibrillation detecting device configured to:
detect the marker of atrial fibrillation.

18. The method of claim 15, wherein atrial fibrillation is monitored by an external device configured to:
responsive to patient activation, detect the marker of atrial fibrillation.

19. The method of claim 16, wherein a notification of a qualifying event is configured to:
track a patient written note; and
send the patient written note to the physician and the patient health record database.

20. The method of claim 16, wherein a notification of a qualifying event is configured to:
store qualifying events and patient compliance with recommended therapy in the patient health record database.

21. The method of claim 19, wherein the atrial fibrillation detecting device is further comprising:
an activity tracker configured to:
collect a plurality of activity data, and
store the plurality of activity data, and
a heart rate tracker configured to:
collect a plurality of heart rate data, and
store the plurality of heart rate data; wherein the atrial fibrillation detecting device is further configured to:
determine the subject's adherence to wearing the atrial fibrillation sensing smart watch based on the plurality of activity data and the plurality of heart rate data; and
responsive to determining the subject is not adherent, notify a health center.

22. The method of claim 21, wherein the atrial fibrillation detecting device is further configured to perform an ECG.

23. The method of claim 13, wherein the medical intervention is an anticoagulant providing rapid therapeutic onset to reduce the risk of the adverse event, and the anticoagulant is administered by way of at least one of swallowing, inhalation, absorption, and injection.

24. The method of claim 23, wherein the anticoagulant providing rapid therapeutic onset comprises at least one of apixaban, betrixaban, edoxaban, otamixaban, razaxaban, rivaroxaban.

25. The method of claim 13, wherein the adverse event is an atrial fibrillation related stroke.

26. The method of claim 13, wherein the adverse event is an atrial fibrillation related systemic embolization.

27. The method of claim 13, wherein the adverse event is an atrial fibrillation related mortality.

28. The method of claim 13, wherein the medical intervention is intermittently administered to prevent a second adverse event.

29. The method of claim 13, wherein the calculated time period of risk is based on the detection of a marker, the method further comprising:
monitoring the patient during the administration of the medical intervention in order to detect the marker;
determining if the marker exceeds the predetermined threshold;
responsive to the determining the marker exceeds the predetermined threshold during the medical intervention, restarting the calculated time period of risk for the medical intervention to be administered for.
